(19) United States
(12) Patent Application Publication
(10) Pub. No.: US 2025/0134486 A1
(43) Pub. Date: May 1, 2025

(54) STETHOSCOPE

(71) Applicant: Tymphany HK Limited, Hong Kong (HK)

(72) Inventor: Chao-An LIN, Taipei City (TW)

(21) Appl. No.: 18/799,595

(22) Filed: Aug. 9, 2024

Related U.S. Application Data
(60) Provisional application No. 63/594,783, filed on Oct. 31, 2023.

(51) Int. Cl. A61B 7/02

(52) U.S. Cl. CPC A61B 7/02

(57) ABSTRACT

A stethoscope includes a tube and a sound conducting assembly. The tube is formed by a plastic injection molding process or a thermal forming process. The sound conducting assembly is disposed in the tube. The tube is completely formed by another plastic injection molding process or another thermal forming process to enclose the sound conducting assembly. The stethoscope is directly obtained in the injection molding machine, and the step of inserting the sound conducting assembly through the tube is not needed.

### CROSS-REFERENCE TO RELATED APPLICATIONS

This application claims priority of Provisional Application No. 60/594,783, filed on Oct. 31, 2023 under 35 USC § 119 (e), the entire contents of which is hereby incorporated herein by reference.

### BACKGROUND OF THE INVENTION

### Field of the Invention

The invention relates to a technical field of medical equipment, and more particularly to a stethoscope and a manufacturing method thereof.

### Description of the Related Art

A conventional stethoscope includes a hollow tube and a sound conducting assembly. When the stethoscope is manufactured, the hollow tube is formed by dipping a mold into liquid PVC, and afterwards all components of the sound conducting assembly are assembled into the tube.

The conventional manufacturing method of the stethoscope has the following problems: (1) a longer time is needed for assembly of the hollow tube and the sound conducting assembly; and (2) the hollow tube may have uneven thickness of tube wall due to the manufacturing method, which seriously influences the appearance and the quality of the stethoscope.

### BRIEF SUMMARY OF THE INVENTION

The invention provides a stethoscope and a manufacturing method thereof, which solves the problems of the longer time for assembly, the low efficiency in assembly and poor quality of product occurring in the conventional stethoscopes.

The stethoscope in accordance with an exemplary embodiment of the invention includes a tube and a sound conducting assembly, the tube is formed by a plastic injection molding process or a thermal forming process. The sound conducting assembly is disposed in the tube, wherein the tube encloses the sound conducting assembly by another plastic injection molding process or another thermal forming process.

In another exemplary embodiment, the tube comprises a main body, a first output tube and a second output tube, one end of the main body is connected to one end of the first output tube and one end of the second output tube.

In yet another exemplary embodiment, the main body, the first output tube and the second output tube are connected to form a Y-shaped tube, the tube further comprises a connecting arm with one end connecting to the first output tube and the other end connecting to the second output tube.

In another exemplary embodiment, the connecting arm is a solid arm made by a plastic injection molding process or a thermal forming process.

In yet another exemplary embodiment, the tube comprises an upper tube body and a lower tube body, the upper tube body comprises an upper portion of the main body, an upper portion of the first output tube and an upper portion of the second output tube, the upper portion of the main body, the upper portion of the first output tube and the upper portion of the second output tube are integrally formed, the lower tube body comprises a lower portion of the main body, a lower portion of the first output tube and a lower portion of the second output tube, the lower portion of the main body, the lower portion of the first output tube and the lower portion of the second output tube are integrally formed.

In another exemplary embodiment, the sound conducting assembly further comprises a sound conducting wire, and the tube firmly encloses an outer surface of the sound conducting wire.

In yet another exemplary embodiment, the sound conducting assembly further comprises a first output metal tube and a second output metal tube, a portion of the first output metal tube engaging the other end of the first output tube, and a portion of the second output metal tube engaging the other end of the second output tube, and one end of the sound conducting wire extends out of the tube through the first output metal tube and the second output metal tube.

In another exemplary embodiment, the sound conducting assembly further comprises a third output metal tube, a portion of the third output metal tube engages the other end of the main body, and one end of the sound conducting wire extends out of the tube through the third output metal tube.

In yet another exemplary embodiment, the tube has a cross section having a diameter ranging from 2 mm to 15 mm.

The manufacturing method of stethoscope in accordance with an exemplary embodiment of the invention includes: forming a portion of a tube through a plastic injection molding process or a thermal forming process, wherein the tube has a chamber; placing a sound conducting assembly into the chamber; and forming the tube enclosing the sound conducting assembly through another plastic injection molding process or another thermal forming process.

In another exemplary embodiment, the tube comprises an upper tube body and a lower tube body, and forming the portion the tube through the plastic injection molding process or the thermal forming process further comprises forming the lower tube body through the plastic injection molding process or the thermal forming process.

In yet another exemplary embodiment, placing the sound conducting assembly into the chamber further comprises placing the sound conducting assembly into the lower tube body, wherein the chamber is a hollow portion of the lower tube body.

In another exemplary embodiment, forming the tube enclosing the sound conducting assembly comprises forming the upper tube body of the tube through another plastic injection molding process or another thermal forming process, wherein the upper tube body and the lower tube body firmly enclose the sound conducting assembly.

In yet another exemplary embodiment, the tube comprises a main body, a first output tube and a second output tube, the upper tube body comprises an upper portion of the main body, an upper portion of the first output tube and an upper portion of the second output tube, the lower tube body comprises a lower portion of the main body, a lower portion of the first output tube and a lower portion of the second output tube, and the main body, the first output tube and the second output tube are connected to form a Y-shaped tube.

In another exemplary embodiment, the tube further comprises a connecting arm, the lower tube body comprises a lower portion of the connecting arm, one end of the lower portion of the connecting arm is connected to the lower portion of the first output tube, and the other end of the lower portion of the connecting arm is connected to the lower portion of the second output tube, the upper body comprises an upper portion of the connecting arm, one end of the upper portion of the connecting arm is connected to the upper portion of the first output tube, and the other end of the upper portion of the connecting arm is connected to the upper portion of the second output tube.

In the embodiments of the present invention, the sound conducting assembly is disposed into the sound conduction tube, and the sound conduction tube encloses the sound conducting assembly through another plastic injection molding process or another thermal forming process. Therefore, the stethoscope is formed in the plastic injection molding machine, and placing the sound conducting assembly into the tube is not needed, whereby the time for assembly is reduced, and the production efficiency is increased. The tube is manufactured through a plastic injection molding process instead of conventional PVC dip coating process, and the conventional hollow tube is replaced by the tube enclosing the sound conducting assembly, whereby the wall thickness of the tube is controlled to be even, the product quality is thus promoted and the environment pollution is also reduced.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 is a perspective view of an embodiment of a stethoscope of the present invention;

FIG. 2 is an exploded view of an embodiment of a stethoscope of the present invention; and

FIG. 3 is a flow chart of an embodiment of a stethoscope of the present invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

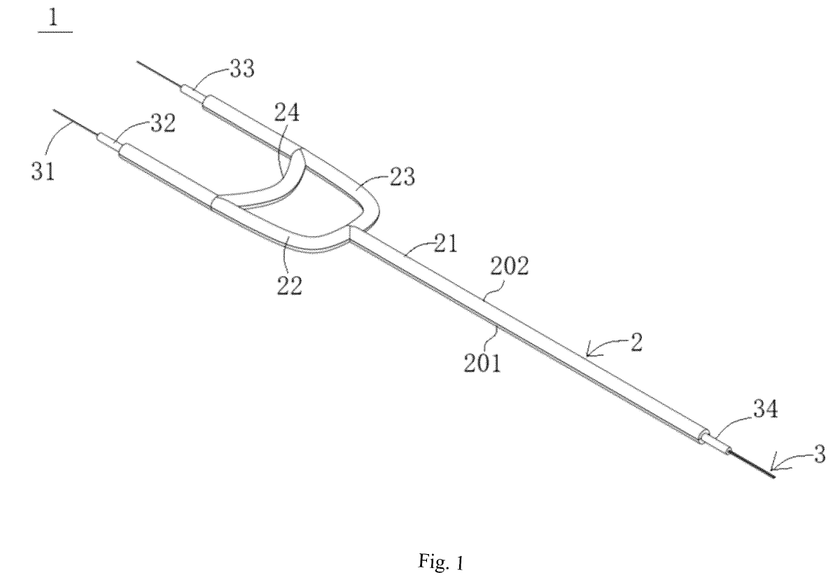

Referring to FIG. 1, an embodiment of a stethoscope of the present invention is disclosed. A stethoscope 1 of the present embodiment is applied for an electronic stethoscope to simplify the assembly thereof. In the present embodiment, the stethoscope 1 includes a tube 2 and a sound conducting assembly 3. The tube 2 is formed by a plastic injection molding process or a thermal forming process. For example, in the plastic injection molding process, a thermoset rubber or thermoplastic rubber is injected into a mold of a tube for plastic injection molding. The sound conducting assembly 3 is disposed in a chamber of the tube 2. The sound conducting assembly 3 becomes enclosed by the tube 2 in another plastic injection molding process or another thermal forming process. In other words, the sound conducting assembly 3 is placed in a mold in which the tube is formed, another plastic injection molding process or another thermal forming process is performed, and afterwards the tube 2 encloses the sound conducting assembly 3, wherein no chamber exists between the tube 2 and the sound conducting assembly 3. The tube 2 firmly encloses the sound conducting assembly 3. Therefore, the stethoscope 1 is directly obtained from the molding process for forming the tube, and no step of inserting the sound conducting assembly 3 through the tube 2 is needed, whereby time for assembly is reduced and the production efficiency is increased. The tube 2 is manufactured through a plastic injection molding process or a thermal forming process instead of a conventional PVC dip coating process, and the conventional hollow tube 2 is replaced by the tube 2 enclosing the sound conducting assembly, whereby the wall thickness of the tube 2 is controlled to be even, the product quality is thus promoted and the environment pollution is also reduced.

Referring to FIG. 1 again, the tube 2 includes a main body 21, a first output tube 22 and a second output tube 23. One end of the main body 21 is connected to one end of the first output tube 22 and one end of the second output tube 23. The main body 21, the first output tube 22 and the second output tube 23 are connected to form a Y-shaped tube. The sound conducting assembly 3 is disposed in the Y-shaped tube, and the two ends from one side of the sound conducting assembly 3 extend out of the Y-shaped tube through the first output tube 22 and the second output tube 23 and connect to a hearing portion (not shown), such as ear pieces. The other end of the sound conducting assembly 3 extends out of the Y-shaped tube through the main body 21 and connects to a sound collection portion (not shown), such as a chest piece.

The tube 2 further includes a connecting arm 24. One end of the connecting arm 24 is connected to the first output tube 22, and the other end of the connecting arm 24 is connected to the second output tube 23. The connecting arm 24 is a solid arm formed by a plastic injection molding process or a thermal forming process. In a plastic injection molding process, thermoset rubber or thermoplastic rubber is injected into a mold to form the tube 2 as well as form the connecting arm 24. When the stethoscope 1 is used, a certain clamping force must exist between the first output tube 22 and the second output tube 23. A conventional stethoscope includes a metal clamping member disposed between the first output tube and the second output tube to provide clamping force therebetween. However, in the present embodiment, the connecting arm 24 is disposed between the first output tube 22 and the second output tube 23. The elastic force generated by the connecting arm 24 made of rubber is utilized as the clamping force as generated by the conventional metal clamping member, whereby the clamping effect is enhanced, the manufacturing process is simplified and the manufacturing cost is reduced.

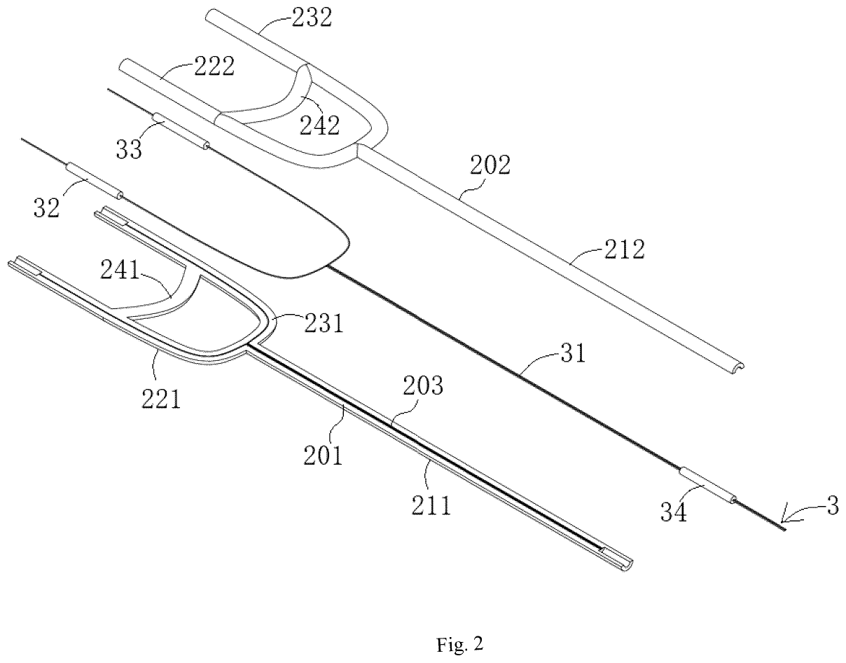

Referring to FIG. 1 again, the tube 2 has a cross section having a diameter ranging from 2 mm to 15 mm. The tube 2 includes a lower tube body 201 and an upper tube body 202. Referring to FIG. 2, the lower tube body 201 includes a lower portion 211 of the main body 21, a lower portion 221 of the first output tube 22 and a lower portion 231 of the second output tube 23. In the present embodiment, the lower portion 211 is a lower half portion of the main body 21 with respect to an axial line horizontally extending through the main body 21. The lower portion 221 is a lower half portion of the first output tube 22 with respect to the axial line horizontally extending through the first output tube 22. The lower portion 231 is a lower half portion of the second output tube 23 with respect to the axial line horizontally extending through the second output tube 23. The lower portion 211 of the main body 21, the lower portion 221 of the first output tube 22 and the lower portion 231 of the second output tube 23 are integrally formed. In a plastic injection molding process, thermoset rubber or thermoplastic rubber is injected into a mold to form the lower tube body 201. The upper tube body 202 includes an upper portion 212 of the main body 21, an upper portion 222 of the first output tube 22 and an upper portion 232 of the second output tube 23. The upper portion 212 is an upper half portion of the main body 21 with respect to the axial line horizontally extending through the main body 21. The upper portion 222 is an upper half portion of the first output tube 22 with respect to the axial line horizontally extending through the first output tube 22. The upper portion 232 is an upper half portion of the second output tube 23 with respect to the axial line horizontally extending through the second output tube 23. The upper portion 212 of the main body 21, the upper portion 222 of the first output tube 22 and the upper portion 232 of the second output tube 23 are integrally formed. In another plastic injection molding process, the sound conducting assembly 3 is placed into the lower tube body 201 already formed in the mold, and afterwards the thermoset rubber or thermoplastic rubber is injected into the mold again to form the upper tube body 202, whereby the upper tube body 202 and the lower tube body 201 encloses the sound conducting assembly 3.

Referring to FIGS. 1 and 2, the sound conducting assembly 3 includes a sound conducting wire 31. Preferably, the tube 2 firmly encloses an outer surface of the sound conducting wire 31, and no space (no gap) exists between the tube 2 and the sound conducting wire 31. The sound conducting assembly 3 further includes a first output metal tube 32 and a second output metal tube 33. A portion of the first output metal tube 32 engages the other end of the first output tube 22, and a portion of the second output metal tube 33 engages the other end of the second output tube 23, and the two ends from one side of the sound conducting wire 31 extend out of the tube 2 through the first output metal tube 32 and the second output metal tube 33. The sound conducting assembly 3 further includes a third output metal tube 34, a portion of the third output metal tube 34 engages the other end of the main body 21, and the other end of the sound conducting wire 31 extends out of the tube 2 through the third output metal tube 34. In the present embodiment, the sound conducting wire 31 is a Y-shaped wire corresponding to the Y-shaped tube, wherein the main body 21, the first output tube 22 and the second output tube 23 enclose the outer surface of the Y-shaped wire. The two ends from the same side of the Y-shaped wire extend out of the first output tube 22 through the first output metal tube 32 and extend out of the second output tube 23 through the second output metal tube 33 to connect the hearing portion. The other end of the Y-shaped wire extends out of the main body 21 to connect the sound collection portion. Moreover, the sound conducting assembly 3 may include other components according to requirement.

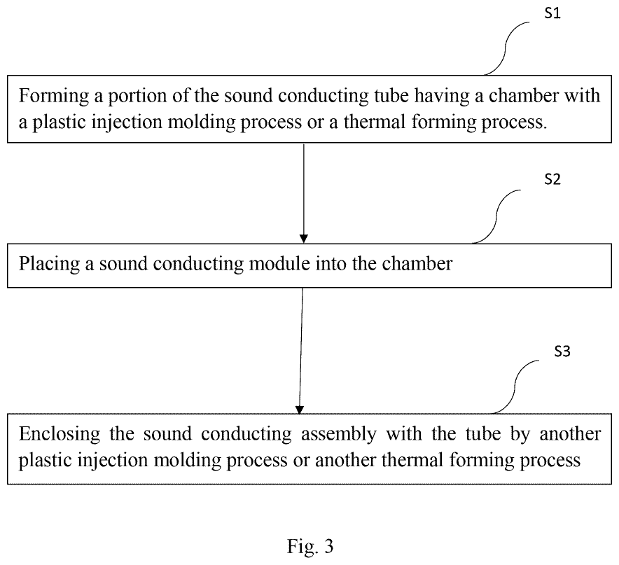

Referring to FIG. 3 and also referring to FIGS. 1 and 2, an embodiment of a manufacturing method of the stethoscope of the present invention is disclosed. The manufacturing method includes steps S1, S2 and S3. First in the step S1, a plastic injection molding process or a thermal forming process is used to form a portion of the tube 2 having a chamber 203. In the first plastic injection molding process, the thermoset rubber or thermoplastic rubber is injected into the mold to form a tube 2 provided with a chamber 203, which can be carried out by a thermal forming process. In the step S2, the sound conducting assembly 3 is placed into the chamber 203. Finally, in the step S3, another plastic injection molding process or another thermal forming process is performed to form a complete tube 2 enclosing the sound conducting assembly 3. In the second plastic injection molding process, the thermoset rubber or thermoplastic rubber is injected into the mold to form the complete tube 2 enclosing the sound conducting assembly 3 in a solid manner. The aforementioned steps can also be completed by the thermal forming process. In the present embodiment, a portion of the tube 2 having a groove as a chamber 203 is formed by the injection molding process, some inner components (the sound conducting assembly 3) are placed into the groove, and finally the complete tube 2 is formed by another injection molding process. The material for the injection molding process is thermoset rubber or thermoplastic rubber. In other embodiment, the manufacturing method is carried out by the thermal forming process, whereby a portion or a half of the product is formed. The components are placed into the half product, and afterwards an injection molding process is used to form the complete product. The final process can also be a thermal forming process. The present embodiment utilizes two injection molding processes to perform the tube 2 enclosing the sound conducting assembly 3, which replaces the conventional process for the assembly of a stethoscope to promote the quality of product and the efficiency of manufacturing.

Referring to FIGS. 1 and 2 again, the tube 2 includes a lower tube body 201 and an upper tube body 202. A lower tube body 201 is a lower half portion of the tube 2 with respect to the axial line extending horizontally through the tube 2. The tube 2 includes the main body 21, the first output tube 22 and the second output tube 23. The main body 21, the first output tube 22 and the second output tube 23 are connected to form a Y-shaped tube. The lower tube body 201 includes the lower portion 211 of the main body 21, the lower portion 221 of the first output tube 22 and the lower portion 231 of the second output tube 23. The tube 2 further includes a connecting arm 24. The lower tube body 201 includes a lower portion 241 of the connecting arm 24. One end of the lower portion 241 of the connecting arm 24 is connected to the lower portion 221 of the first output tube 22, and the other end of the lower portion 241 of the connecting arm 24 is connected to the lower portion 231 of the second output tube 23. In the step S1, an injection molding process is used to form the lower tube body 201.

In the step S2, the sound conducting assembly 3 is placed into the lower tube body 201, wherein the chamber 203 is a hollow portion of the lower tube body 201. In the step S3, another plastic injection molding process or another thermal forming process is used to form the upper tube body 202. The upper tube body 202 and the lower tube body 201 solidly enclose the sound conducting assembly 3. The upper tube body 202 is an upper half portion of the tube 2 with respect to the axial line extending horizontally through the tube 2. The upper tube body 202 includes the upper portion 212 of the main body 21, the upper portion 222 of the first output tube 22 and the upper portion 232 of the second output tube 23. The upper tube body 202 further includes the upper portion 242 of the connecting arm 24. One end of the upper portion 242 of the connecting arm 24 is connected to the upper portion 222 of the first output tube 22, and the other end of the upper portion 242 of the connecting arm 24 is connected to the upper portion 232 of the second output tube 23.

The present invention discloses a stethoscope and a manufacturing method thereof. A portion of the tube provided with a groove is formed by injection molding process. The inner components are put into the groove, and finally a complete tube is formed by another injection molding process. The material for injection molding process can be thermoset rubber or thermoplastic rubber. The manufacturing method is also performed by the thermal forming process, whereby a portion or a half of the product is formed. The components are put into the half product, and afterwards an injection molding process is used to form the complete product. The final process can also be a thermal forming process. Therefore, the stethoscope is formed in the plastic injection molding machine, and the step of inserting the sound conducting assembly through the tube is not needed, whereby the time for assembly is reduced, and the production efficiency is increased. The tube is manufactured through plastic injection molding process instead of conventional PVC dip coating process, and the conventional hollow tube is replaced by the tube enclosing the sound conducting assembly, whereby the wall thickness of the tube is controlled to be even, the product quality is thus promoted and the environment pollution is also reduced.

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A stethoscope, comprising:
a tube formed by a plastic injection molding process or a thermal forming process; and
a sound conducting assembly disposed in the tube,
wherein the tube encloses the sound conducting assembly by another plastic injection molding process or another thermal forming process.

2. The stethoscope as claimed in claim 1, wherein the tube comprises:
a first output tube;
a second output tube; and
a main body having one end connected to one end of the first output tube and one end of the second output tube.

3. The stethoscope as claimed in claim 2, wherein the main body, the first output tube and the second output tube are connected to form a Y-shaped tube, and
wherein the tube further comprises a connecting arm with one end connecting to the first output tube and the other end connecting to the second output tube.

4. The stethoscope as claimed in claim 3, wherein the connecting arm is a solid arm made by a plastic injection molding process or a thermal forming process.

5. The stethoscope as claimed in claim 2, wherein the tube comprises:
an upper tube body comprising:
an upper portion of the main body;
an upper portion of the first output tube; and
an upper portion of the second output tube, the upper portion of the main body; and
a lower tube body comprising:
a lower portion of the main body;
a lower portion of the first output tube; and
a lower portion of the second output tube
wherein the upper portion of the main body, the upper portion of the first output tube and the upper portion of the second output tube are integrally formed, and
wherein the lower portion of the main body, the lower portion of the first output tube and the lower portion of the second output tube are integrally formed.

6. The stethoscope as claimed in claim 2, wherein the sound conducting assembly further comprises a sound conducting wire, and the tube encloses an outer surface of the sound conducting wire.

7. The stethoscope as claimed in claim 6, wherein the sound conducting assembly further comprises:
a first output metal tube; and
a second output metal tube,
wherein a portion of the first output metal tube engages another end of the first output tube,
wherein a portion of the second output metal tube engages another end of the second output tube, and
wherein two ends of the sound conducting wire extend out of the tube through the first output metal tube and the second output metal tube.

8. The stethoscope as claimed in claim 6, wherein the sound conducting assembly further comprises a third output metal tube,
wherein a portion of the third output metal tube engages another end of the main body, and
wherein one end of the sound conducting wire extends out of the tube through the third output metal tube.

9. The stethoscope as claimed in claim 1, wherein the tube has a cross section having a diameter ranging from 2 mm to 15 mm.

10. A manufacturing method of stethoscope, comprising:
forming a portion of a tube through a plastic injection molding process or a thermal forming process, wherein the tube has a chamber;
placing a sound conducting assembly into the chamber; and
forming the tube to enclose the sound conducting assembly through another plastic injection molding process or another thermal forming process.

11. The manufacturing method as claimed in claim 10, wherein the tube comprises:
an upper tube body; and
a lower tube body, and
wherein said forming the portion the tube through the plastic injection molding process or the thermal forming process comprises forming the lower tube body through the plastic injection molding process or the thermal forming process.

12. The manufacturing method as claimed in claim 11, wherein said placing the sound conducting assembly into the chamber comprises placing the sound conducting assembly into the lower tube body, and
wherein the chamber is a hollow portion of the lower tube body.

13. The manufacturing method as claimed in claim 12, wherein said forming the tube to enclose the sound conducting assembly comprises forming the upper tube body of the tube through another plastic injection molding process or another thermal forming process, and
wherein the upper tube body and the lower tube body enclose the sound conducting assembly.

14. The manufacturing method as claimed in claim 13, wherein the tube comprises:
a main body;
a first output tube; and
a second output tube,
wherein the upper tube body comprises:
an upper portion of the main body;
an upper portion of the first output tube; and
an upper portion of the second output tube,
wherein the lower tube body comprises:
a lower portion of the main body;
a lower portion of the first output tube; and
a lower portion of the second output tube, and
wherein the main body, the first output tube and the second output tube are connected to form a Y-shaped tube.

15. The manufacturing method as claimed in claim 14, wherein the tube further comprises a connecting arm,
wherein the lower tube body comprises a lower portion of the connecting arm, one end of the lower portion of the connecting arm being connected to the lower portion of the first output tube and another other end of the lower portion of the connecting arm being connected to the lower portion of the second output tube, and
wherein the upper body comprises an upper portion of the connecting arm, one end of the upper portion of the connecting arm being connected to the upper portion of the first output tube and another other end of the upper portion of the connecting arm being connected to the upper portion of the second output tube.
